(19) United States
(12) Patent Application Publication
(10) Pub. No.: US 2022/0183580 A1
(43) Pub. Date: Jun. 16, 2022

(54) METHOD OF PROVIDING SPOKEN INSTRUCTIONS FOR A DEVICE FOR DETERMINING A HEARTBEAT

(71) Applicant: Happitech B.V., Amsterdam (NL)

(72) Inventors: Yosef SAFI-HARB, Amsterdam (NL); Jonas Stephan Sebastiaan Gabriel DE JONG, Amsterdam (NL)

(21) Appl. No.: 17/396,119

(22) PCT Filed: Feb. 7, 2019
(86) PCT No.: PCT/NL2019/050080

§ 371 Date: Aug. 6, 2021

(51) Int. Cl. A61B 5/024; A61B 5/00

(52) U.S. Cl. CPC A61B 5/02416; A61B 5/02438; A61B 5/7455; A61B 5/7405; A61B 5/742; A61B 5/7221

(57) ABSTRACT

In a handheld device for determining heartbeat/heart rhythm of a user, a method is provided for providing spoken instructions for the user of the device. The method comprises obtaining, over time, via an optical sensor like a camera, a data signal and providing the data signal to an electronic data processor. In the electronic data the processor, determining, based on the signal, a quality factor for the signal. In the electronic data processor, an algorithm is executed for determining a heartbeat using data comprised by the data signal as input. The electronic data processor looks up, from an electronic memory, at least one audio-visual data file comprising data representing audio-visual feedback instructions, based on at least one of the quality factor and the outcome of the execution of the algorithm. A speaker is used to reproduce the audio-visual feedback instructions and aid the user in device placement.

### TECHNICAL FIELD

The various aspects and examples thereof relate to providing audible instructions and feedback and spoken messages in particular to a user of a handheld device for determining a heartrate.

### BACKGROUND

Determining a heartrate and heart rhythm (also know as pulse rate pulse rhythm) by processing data obtained by placing a finger on a sensor, aided by a light source, is known. Such measurements provide data reliable enough for monitoring one's heartrate for recreational purposes, for healthy people. Such measurements may be done using dedicated photoplethysmographic (PPG) sensors. Such sensors may be placed on fingers of patients for a longer time, for example while they are hospitalized—or outside of hospital—and need to be monitored for a longer time. In this way, accurate data may be obtained by using data obtained over a longer period of time.

If heartrate or heart rhythm is determined using a handheld device, like a smartphone, a long data acquisition period is not feasible (or at least uncomfortable). This may be an issue for obtaining accurate data. Reasons may be that the device is handheld and that users usually do not want the measurement to take a serious amount of time. For recreational or amateur use of the heartrate data, this may not be an issue. However, if the data obtained by means of the handheld consumer device is to be used for medical purposes, this is an issue.

### SUMMARY

Hence, it is preferred to provide a method for providing more accurate measurements. Using motion compensation is a relatively common technique to compensate for motion of a handheld device when acquiring data, for example when taking a photograph by means of a camera comprised by a smartphone, but that solution has a lot of room for improvement when obtaining optical data for determining a heartrate, heart rhythm or pulse rate. Therefore, other solutions are required.

To a medically skilled person, a heartrate and a pulse rate may be different: a heart may give one beat without actually pumping blood, in which no pulse is generated. Yet, for this document, the terms heart beat and pulse beat may be used interchangeably and the same applies to the term heart rate and pulse rate, heart rate variability and pulse rate variability, heart rhythm and pulse rhythm: for this document, they have the same function. Furthermore, it is noted that a heart rate or pulse rate is an average amount of pulses per time unit and that heart rhythm or pulse rhythm is the actual cadence of pulses as a function of time. Hence, heart rhythm and heart rate are different entities, both based on detected pulses or beats—of the heart or of blood through one or ore body vessels.

A first aspect provides, in a handheld device for determining at least one of a heartbeat or a/heart rhythm/of a user, a method of providing spoken instructions for the user of the device. The method comprises obtaining, over time, via an optical sensor fixed to the handheld device and in proximity of a member of the body of the user, a data signal and providing the data signal to an electronic data processor. The method further comprises, in the electronic data the processor, determining, based on the signal, a quality factor for the signal and executing an algorithm for determining at least one of the heartbeat and the heart rhythm using data comprised by the data signal as input. The method also comprises looking up, by the electronic data processor and from an electronic memory, at least one audio-visual data file comprising data representing at least one of audio-visual and haptic feedback instructions, based on at least one of the quality factor and the outcome of the execution of the algorithm and, using a speaker or another actuator, reproducing the at least one of audio-visual and, haptic feedback instructions. The speaker may be comprised by the device, but may also be an external speaker connected to the device over a wired or wireless connection; in that case, audio data is rendered by the device and sent to the speaker.

By virtue of this functionality, a user of the device is enabled to execute measurements with respect to their heartbeats and obtain any data related thereto in a more accurate way. The improved accuracy is provided by evaluating the quality of the signal and providing instructions, for example by means of spoken feedback, to position himself of herself or to position a body part differently relative to the device. Alternatively or additionally, the instructions may also relate to placement of the device. In the latter case, rather than the quality factor of the signal, position of the device may be determined and serve as an input.

By providing spoken feedback or other feedback by means of the speaker or vibrator, the user does not have to pay attention to a screen that may be comprised by the device and can fully focus on positioning the device and their body or a body part relative to one another.

The device may be a mobile telephone of any kind, a personal digital assistant, an electronic watch having the appropriate sensor capabilities or any other type of wearable or handheld electronic devices having the appropriate sensor capabilities.

An embodiment further comprises receiving, via an electronic input module of the handheld device, instructions for starting a procedure for determining a heartbeat, looking up, in the electronic memory, at least one audio-visual data file comprising data representing audio-visual handling instructions providing the user instruction to prepare for acquisition of the data signal; and using a speaker, reproducing the at least one of audio-visual and haptic handling instructions.

This embodiment may be used to fully guide a user of the device through a procedure for obtaining a heartrate (pulse rate) and/or heart rhythm (or pulse rhythm).

Another embodiment, wherein a further optical sensor is fixed to the handheld device, comprises determining capabilities of at least one of the optical and motion sensor and the further optical sensor, determine whether capabilities of at least one of the optical sensor and the further optical sensor are sufficient for providing data as input for the algorithm to successfully determine a heartbeat, obtaining position data for the handheld device; and determine, based on the position data, whether in the determined position of the handheld device, an optical sensor is exposed having capabilities sufficient for providing data as input for the algorithm to successfully determine a at least one of a heartbeat or a heart rhythm. In this embodiment, the audio-visual handling instructions instruct the user to position the handheld to expose an optical sensor having capabilities sufficient for providing data as input for the algorithm to successfully determine a heartbeat if it has been determined that in the determined position no optical sensor is exposed having capabilities sufficient for providing data as input for the algorithm to successfully determine a heartbeat.

Optical devices and mobile telephones have a bar-type form factor in particular may comprise a first camera at the side of the screen and a second camera at the opposite side. These cameras may have different capabilities, like light sensitivity, resolution, colour sensitivity, other, or a combination thereof. For lower end telephones, the front camera—at the screen side—may not have capabilities to sufficiently accurate determine a series of heartbeats. Therefore, the camera at the rear side is preferably used but nonetheless the front camera could also be used. This embodiment provides a method for guiding a user to properly interact with the sensor and/or the camera lens.

Again a further embodiment comprises obtaining motion data by means of a motion sensor, further comprising movement data for the handheld device, wherein the looking up of the audio-visual data file comprising audio-visual data is also based on the motion data.

Movement of the device by the user may influence the results of measurements. With this embodiment, the user may be instructed to lay the device on a stable surface to continue the measurement Yet another embodiment comprises receiving microphone data from a microphone in proximity of the user, extracting an audio data value from the microphone and looking up an audio file comprising data representing audible corrective instructions if the audio data value is outside a pre-determined audio range. The microphone may pick up any audible disturbances and possibly other vibrations that may influence the measurement, including talking of the person under scrutiny. An instruction may be provided to a user not to speak if speaking of a user is detected.

A second aspect provides a handheld device for determining heartbeat of a user, and for providing spoken instructions for the user of the device. The device comprises an optical sensor in proximity of a member of the body of the user for obtaining, over time, a data signal and an electronic data processor. The electronical data processor is arranged to determine, based on the signal, a quality factor for the signal, execute an algorithm for determining a heartbeat using data comprised by the data signal as input, look up, from an electronic memory, at least one audio-visual data file comprising data representing audio-visual feedback instructions, based on at least one of the quality factor and the outcome of the execution of the algorithm. The device further comprises a speaker for, reproducing the audio-visual feedback instructions.

A third aspect provides a computer program product comprising computer executable instructions causing the computer, when the loaded in the memory of the computer, to execute the method according to the first aspect.

### BRIEF DESCRIPTION OF THE DRAWINGS

The various aspects and examples thereof will now be further elucidated in conjunction with drawings. In the drawings:

FIG. 1: shows an example of the device according to the second aspect; and

FIG. 2: shows a flowchart depicting an example of the method according to the first aspect.

### DETAILED DESCRIPTION

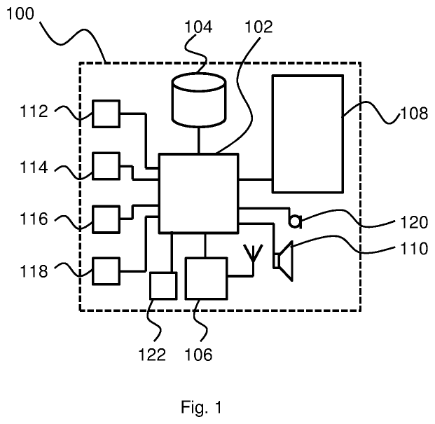

FIG. 1 shows a smartphone 100 as an embodiment of the second aspect. The smartphone 100 comprises a central processing unit 102, a memory module 104, a communication module 106, a screen 108 and a speaker 110. The smartphone 100 further comprises a first camera 112 as a first optical sensor, a second camera 114 as a second optical sensor, a gyroscope 116 as a position sensor and an accelerometer 118 as a motion sensor. The first camera 112 is preferably provided with a light source, preferably a bright light source emitting light in the visible domain, like a blue LED with a phosphorous coating having broad spectrum fluorescent characteristics. It is noted that the smartphone 100 may comprise further sensors for detecting motion and position. Furthermore, the gyroscope 116 may also be used as a motion sensor. In the embodiments discussed here, the first camera 112 is provided at the back of the smartphone 100 and the second camera 114 is provided at the front, at which location also the screen 108 is provided.

The communication module 106 is arranged for communicating with other devices, preferably over radio frequency enable communication protocols like Bluetooth, IEEE 802.11, and 3G/4G/5G and successive and equivalent mobile and in particular cellular communication protocols. The memory module 106 may be a fixed memory, a removable memory like an SDcard, other, or a combination thereof. The screen 108 is preferably a touchscreen for displaying data and receiving user input. Additionally, or alternatively, other input modules may be available, like buttons, knobs, other, or a combination thereof. The speaker 110 may be a sole speaker of a group of multiple speakers. Preferably, all components are provided in a single housing. Yet, some components depicted by FIG. 1 may alternatively or additionally be provided external to the housing and operationally connected to the central processing unit 102 over a wired or wireless communication protocol.

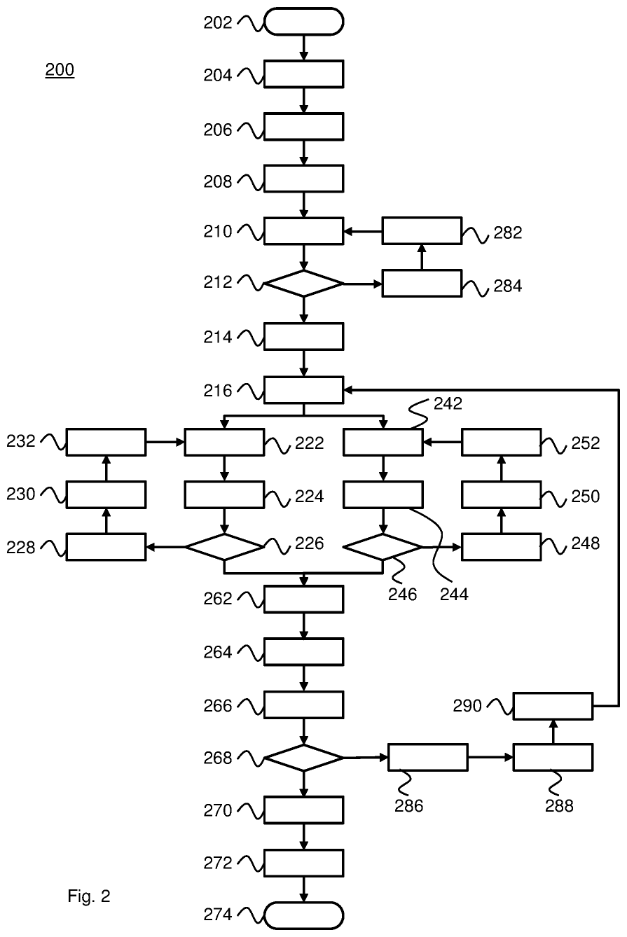

FIG. 2 shows a flowchart 200 depicting a procedure for determining a heart of a person in a reliable way. The various parts of the flow chart 200 are briefly summarised directly below and will be further elucidated after the list.
202 start
204 obtain sensor capability data
206 evaluate sensor capability data
208 obtain position data
210 determine position
212 position ok?
214 switch on light
216 provide spoken instruction
222 obtain optical signal data
224 determine signal quality factor
226 signal quality factor ok?
228 evaluate signal quality factor failure
230 retrieve appropriate audio-visual feedback
232 reproduce feedback
242 obtain motion signal data
244 determine motion quality factor
246 motion quality factor ok?
248 evaluate motion quality factor failure
250 retrieve appropriate audio-visual feedback
252 reproduce feedback
262 determine heartbeats from optical signal
264 determine heartrate
266 determine further state data
268 heart rate and/or other parameters provide logical values?
270 lookup and/or synthesise audio-visual feedback data
272 reproduce audio-visual feedback
274 end

The procedure starts in a terminator 202 and continues to step 204 in which sensor capability data is acquired in step 204. The sensor information comprises capabilities of the first camera module 112 and the second camera module 114. Such sensor capability data may provide information on resolution of the cameras, colour capabilities, including colour range and resolution, location on the smartphone 100, light sensitivity data, including actual active sensitivity and sensitivity ranges, whether a light source is provided for illuminating objects that may be captures by the camera, other, or a combination thereof.

The sensor capability data is evaluated in step 206. The outcome of the evaluation may be whether a camera may be capable for providing heartbeat, heart rate variability, heart rhythm, or heartrate measurements in a medically relevant manner and with a proper accuracy for medical applications. Alternatively, other standards may be used. If more available cameras are available, a most applicable camera may be selected. Such selection may be based on the parameters provided above, other, or a combination thereof. The selection may form part of the evaluation.

In step 208, position data of the smartphone 100 is determined. To this end, data may be acquired by means of the gyroscope 116 and the accelerometer 118. Alternatively or additionally, the cameras may be used. If the first camera 112 does not receive light, it may be determined that the smartphone 100 is placed on an opaque surface, the front side or backside facing up. Determining whether the second camera 114 receives light may also be obtained as position data—if one camera receives light and the other does not, the non-light-receiving camera may be facing upward or downward, with the light receiving camera being ready for collecting data and generating a signal based on the received data.

Using the position data, the position of the telephone may be determined. Relevant in this context may be determining what camera may be facing up and what camera may be facing down, lying on a surface. If the smartphone 100 is not moving—which may be determined using data from the gyroscope 116 and the accelerometer 118—and only one camera is receiving light, the smartphone 100 may be assumed to be lying ion a particular surface. If the smartphone 100 is moving, it may be determined to be hand-held.

In step 212, it is determined whether the smartphone 100 is held in a correct position, ready for acquiring sensor data to be used for determining heartrate, heartrate variability and heart rhythm disorders (such as atrial fibrillation for example). The requirements may depend on user data. For example, a ninety-year old or a nine-year old may be required to leave the smartphone 100 sitting on a steady surface, whereas a 25-year old person may be allowed to take measurements while holding the smartphone in the hand. If it is determined that the position of the smartphone 100 is not in a correct position, the central processing unit 102 retrieves audio-visual data from the memory 106. The audio-visual data, for example provided in a file, comprises audio-visual data instructing a user to place the smartphone 100 in a correct position. In particular audible data—sound—feedback may be relevant, if the sensor to be used is a camera at the back side of the smartphone 100. In such case, the screen 108 is to be placed facing down. In that position, information on the screen cannot be viewed by the user—which requires spoken instructions.

Prior to taking measurement, a light source provided with a selected camera may be switched on in step 214. Additionally, a spoken message may be provided instructing the user to place his or her finger on the selected and exposed camera in step 216—the first camera 112. In an alternative embodiment, the light is switched on once the central processing unit 102 determines, based on a signal from the applicable camera, that a finger—or other body part—is placed on the camera.

Subsequently, the process may branch in two sections. In a left branch, in step 222, a video stream comprising consecutive frames is obtained by the first camera 112 as an example of an optical signal received by an optical sensor. The video steam is provided to the central processing unit 102 for evaluation of an optical quality factor. The optical quality factor may be determined based on values of one or more individual frames, a trend of variation of values over multiple frames or both.

With respect to data obtained from one frame captured by the first camera 112, it may be detected that no finger is present at all, depending on colour data—to little red colour available, for example. It may be detected that a finger is pressed too hard on the first camera, if the frame is too dark. If parts of the frame are significantly lighter than other parts, it may be detected that the finger is placed incorrectly on the first camera 112, for example on only half the first camera 112. With respect to values determined from variations from frame to frame and within the frame, it may be determined that a finger is being moved too much to provide a proper measurement. Based on these consequences of incorrect placement of a finger, one or more optical signal quality factors are determined.

The optical signal quality factors are evaluated in step 226 and if the quality factors are within specification, the procedure continues to determining heartbeats in step 262. However, if one or more of the optical signal quality factor or the quality factors are out of specification, the procedure branches to step 248. In step 248, the central processing unit 102 determines what the cause may be for the one or more optical signal quality factors being out of specification. Based on a failure cause determined in step 248, a particular file comprising audio-visual data representing a feedback message, is retrieved by the central processing unit 102 from the memory module 104 in step 250. In step 252, the audio-visual data is reproduced by means of the speaker 110 in case of only audible feedback. Additionally, the audio-visual data may be reproduced using the screen 108—although it will be appreciated this is of little use with the screen 108 facing a surface, as the first camera 112 is the rear camera that faces upward. Subsequently, the procedure branches back to step 222 to obtain data by means of the first camera 112 and calculate one or more optical signal quality factors.

In a right branch, viewed from step 216, the procedure continues to step 242; in step 242, motion data is acquired. Motion data is data related to any kind of motion of the user of smartphone 100, the smartphone 100 or both. Such motion data may be recorded using the gyroscope 116 and the accelerometer 118. Additionally, or alternatively, a microphone 120 may be used. And as more telephone touchscreens like the screen 108 are equipped with pressure sensors, also motion data may be acquired using such pressure sensor that may be comprised by the screen 108.

In step 244, one or more motion quality factors are determined, for example calculated, based on one or more signal values received from the various sensors other than the optical sensors and cameras in particular. In one embodiment, one motion quality factor is determined per sensor or per entity. In another embodiment, one single motion quality factor is determined and in yet another embodiment, a motion quality factor is determined based on values for multiple, not necessarily all entities. The one or more motion quality factors are evaluated against one or more pre-determined thresholds, for example threshold ranges in step 246. If the one or more motion quality factors are outside a pre-determined range or otherwise do not comply with pre-determined conditions, the procedure branches to step 248 in which it is determined what may be a reason for the one or more quality factors being out of spec. if a voice is detected by means of the microphone 120, the reason may be that a person is heard talking. If rotation of the smartphone 100 is detected, for example by means of the gyroscope 116, it may be determined that the smartphone 100 is not being held on a flat surface. If the smartphone 100 is detected to quickly move to and fro, for example by means of the accelerometer 118, it may be determined that the user is shaking. And if a too high pressure is detected, it may be determined the user presses too hard on the device.

Depending on the determined non-optimal condition or conditions for determining a heartrate or other heartbeat related parameters, a file comprising audio-visual data representing feedback is looked up by the central processing unit 102 in the memory module 104 in step 250. In step 252, the audio-visual data is reproduced by means of the speaker 110 and optionally by the screen 108. After the reproduction of that data, the process branches back to step 242 to verify whether the feedback has been followed up.

As discussed, in step 262, a heartbeat is determined based on the optical signal received, in this example based on a video stream comprising consecutive frames acquired by means of the first camera 112. If a finger is held steady on the camera 112 and illumination is kept constant, variation in the optical signal acquired is predominantly caused by changes in blood flow through the finger, which changes are predominantly caused by beating of the heart. In this way, a heartbeat may be detected at a top or a valley of a colour value as a function of time. Such colour value may be an average value of all pixels in a frame, an average value of pixels in an area of the frame or another value of colour. A colour value may be a value of one colour component, like red, green or blue, or of a combination of two or three colour values. Other types of analysis may be used additionally or alternatively to determine an extremity in a value related to the optical signal received that may indicate a heartbeat. Step 262 may be repeated several times and/or during a particular point in time for detecting multiple heartbeats and a period of time between subsequent determined heartbeats.

Based on the data determined in step 262, a heartrate or heart rhythm is determined in step 264. A heartrate is the inverse of the average time between heartbeats. Subsequently, or, alternatively, prior to or in parallel to step 262, other parameters are calculated in step 264. Such parameters may be the standard deviation of time between two heartbeats, heartrate variability, breath rate—based on the determined heartrate variability over time—, other parameters of a combination thereof. Some of such parameters provide information on the accuracy of the heartrate value that has been determined. For example, if the standard deviation of the time period between two heartbeats is too high, the determined heartrate value may be considered to be inaccurate. This applies to the heartrate variability as well. If this is too high, the determined heartrate value may be determined as being inaccurate. However, as this may indicate a high breathing rate, for example after exercise, this check may be omitted.

In step 266, optionally further data on the state of the subject under scrutiny may be acquired. Such data may be, without limitation, be age, physical condition, medicine use or consequences thereof, physical state—being for example in rest or performing a workout—time of the day, time of the year, gender, other, or a combination thereof. In this step, also physical capabilities of the user may be obtained, for example whether the user is audibly or visually impaired or has other physical or mental sensory limitations. The way the feedback may be provided may be adapted to these limitations in the sense that visually impaired users may receive haptic and/or audible feedback and audibly impaired user may receive haptic and/or visual feedback.

Also gender and age of the user may be taken into account; it is known that men of a certain age, in particular above the age 75, have a far larger odd of becoming audibly impaired as compared to women in that age range. Furthermore, it has been demonstrated that also visual capabilities of men and women are different.

In step 268, it is checked whether any determined parameter is out of a specified range or otherwise does not comply with any particular condition. The information obtained in step 266 may be taken into account. If this is the case, the procedure branches to step 286 in which step a potential cause of non-compliance is determined. In step 288, a file comprising audio-visual data is retrieved representing feedback, preferably with instructions to remove the cause of non-compliance and preferably instructions how to remove the cause of non-compliance. The audio-visual data is reproduced in step 290 in a fashion as discussed above and the procedure moves to step 216 for acquiring optical data again.

If the data determined and preferably calculated is determined to be compliant, the procedure continues to step 270, in which step audio-visual feedback data is synthesised. Preformed messages or parts thereof may be retrieved from the memory module 104 by the central processing unit 102 and, based on the determined data, complete feedback messages may be formed. For example, a message may be formed with a preformed message part “your heartrate/heart rhythm is” followed by a number of which audio-visual data is retrieved separately. The synthesised messages are reproduced in step 272, after which the procedure ends in step 274.

It is noted that the smartphone may be provided with additional or alternative sensors. Certain smartphones are provided with dedicated photoplethysmographic sensors for determining heartrate/heart rhythm, which may be used in the same way. Furthermore, it may be envisaged that telephones may be coupled to electronic or electromagnetic sensors provided on a body of a person for acquiring ECG data. Such ECG data may not be as accurate as ECG data acquired using hospital grade equipment, but it may be envisaged such equipment may be used to acquire data and deliver a signal suitable to determine a heartrate.

Smartphones are preferably provided with two cameras: a front camera—at the same side as the screen—for video conversations and a rear camera for taking photographs. The rear camera is usually most suited for acquiring data for determining a heartrate. As discussed, in such scenario, the telephone is preferably placed with the screen down on a flat surface—contrary to currently customary practice. And with the screen down, feedback on the screen—currently customary practice as well—is not feasible. Therefore, the audio-visual data retrieved for communicating with the user for providing feedback or instructions, for example to lay down the smartphone 100 with the screen 108 facing a flat surface like a table top, such instructions are preferably provided in an audible manner, with spoken instructions. Video data with visual instructions may be provided as an option, but these may not always be useful if the smartphone is place on a surface with the screen 108 facing the surface. On the other hand, with most smartphones provided with a vibration module like a vibration module 122 comprised by the smartphone 100, it may be feasible to provide the user with feedback by actuating the vibration module 122 with the central processing unit 102. Such vibration as haptic feedback may be provided in addition to or as an alternative to audio-visual feedback.

In summary, in a handheld device for determining heartbeat/heart rhythm of a user, a method is provided for providing spoken instructions for the user of the device. The method comprises obtaining, over time, via an optical sensor like a camera, a data signal and providing the data signal to an electronic data processor. In the electronic data the processor, determining, based on the signal, a quality factor for the signal. In the electronic data processor, an algorithm is executed for determining a heartbeat using data comprised by the data signal as input. The electronic data processor looks up, from an electronic memory, at least one audio-visual data file comprising data representing audio-visual feedback instructions, based on at least one of the quality factor and the outcome of the execution of the algorithm. A speaker is used to reproduce the audio-visual feedback instructions and aid the user in device placement.

In the description above, it will be understood that when an element such as layer, region or substrate is referred to as being “on” or “onto” another element, the element is either directly on the other element, or intervening elements may also be present. Also, it will be understood that the values given in the description above, are given by way of example and that other values may be possible and/or may be strived for.

Furthermore, the invention may also be embodied with less components than provided in the embodiments described here, wherein one component carries out multiple functions. Just as well may the invention be embodied using more elements than depicted in the Figures, wherein functions carried out by one component in the embodiment provided are distributed over multiple components.

It is to be noted that the figures are only schematic representations of embodiments of the invention that are given by way of non-limiting examples. For the purpose of clarity and a concise description, features are described herein as part of the same or separate embodiments, however, it will be appreciated that the scope of the invention may include embodiments having combinations of all or some of the features described. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality.

A person skilled in the art will readily appreciate that various parameters and values thereof disclosed in the description may be modified and that various embodiments disclosed and/or claimed may be combined without departing from the scope of the invention.

It is stipulated that the reference signs in the claims do not limit the scope of the claims, but are merely inserted to enhance the legibility of the claims.

1. In a handheld device for determining at least one of a heartbeat or a heart rhythm of a user, a method of providing spoken instructions for the user of the device, the method comprising:
obtaining, over time, via an optical sensor fixed to the handheld device and in proximity of a member of a body of the user, a data signal and providing the data signal to an electronic data processor;
in the electronic data processor, determining, based on the data signal, a quality factor for the data signal;
in the electronic data processor, executing an algorithm for determining at least one of the heartbeat and the heart rhythm using data comprised by the data signal as input;
looking up, by the electronic data processor and from an electronic memory, at least one audio-visual data file comprising data representing at least one of audio-visual and haptic feedback instructions, based on at least one of the quality factor and the outcome of the execution of the algorithm;
using a speaker, reproducing the at least one of audio-visual and haptic feedback instructions.

2. The method according to claim 1, further comprising:
receiving, via an electronic input module of the handheld device, instructions for starting a procedure for determining a heartbeat;
looking up, in the electronic memory, at least one audio-visual data file comprising data representing at least one of audio-visual and haptic handling instructions providing the user instruction to prepare for acquisition of the data signal; and
using a speaker, reproducing the at least one of audio-visual and haptic handling instructions.

3. The method according to claim 1, wherein a further optical sensor is fixed to the handheld device, the method further comprising:
determining capabilities of at least one of the optical sensor and the further optical sensor;
determining whether capabilities of at least one of the optical sensor and the further optical sensor are sufficient for providing data as input for the algorithm to successfully determine a heartbeat;
obtaining position data for the handheld device; and
determining, based on the position data, whether in a determined position of the handheld device, an optical sensor is exposed having capabilities sufficient for providing data as input for the algorithm to successfully determine at least one of a heartbeat or a heart rhythm;
wherein the audio-visual handling instructions instruct the user to position the handheld to expose an optical sensor having capabilities sufficient for providing data as input for the algorithm to successfully determine a heartbeat if it has been determined that in the determined position no optical sensor is exposed having capabilities sufficient for providing data as input for the algorithm to successfully determine a heartbeat.

4. The method according to claim 1, further comprising obtaining motion data by means of a motion sensor, wherein the looking up of the audio-visual data file comprising data representing at least one of the audio-visual and haptic feedback instructions is also based on the motion data.

5. The method according to claim 1, wherein looking up the audio-visual data file based on at least one of the quality factor and the outcome of the execution of the algorithm comprises:
determining whether the quality factor is within a pre-determined quality range and if the quality factor is outside the pre-determined quality range;
looking up an audio-visual data file comprising data representing audible corrective instructions if the quality factor is outside the pre-determined quality range;
and reproducing, via the speaker, the data representing audible corrective instructions.

6. The method according to claim 1, further comprising:
determining whether execution of the algorithm yields an algorithm value or a set of algorithm values with a pre-determined algorithm range; and
looking up an audio-visual data file comprising data representing audible corrective instructions if the algorithm value is outside the pre-determined algorithm range.

7. The method according to claim 1, wherein determining the quality factor comprises at least one of determining:
a signal noise level;
a signal to noise ratio;
a signal waveform;
a signal autocorrelation;
a signal periodicity; and
a signal variation;
of the data signal over time.

8. The method according to claim 1, wherein determining the quality factor comprises at least one of determining:
a colour value; and
a light intensity value;
camera type;
camera resolution
phone model;
variation in colour value over time;
variation of regions of the frame over time.

9. The method according to claim 8, wherein the data signal comprises data values on an image grid and determining the quality factor comprises determining at least one of an area colour value and an area light intensity value in an area of the grid.

10. The method according to claim 1, further comprising:
receiving microphone data from a microphone in proximity of the user;
extracting an audio data value from the microphone data; and
looking up an audio-visual data file comprising data representing audible corrective instructions if the audio data value is outside a pre-determined audio range.

11. The method according to claim 1, further comprising, during obtaining of the data signal, operating a light source comprised by the handheld device in at least one of a continuous mode and an intermitting mode.

12. The method according to claim 1, further comprising:
receiving movement data from a movement sensor providing information on a movement of the handheld device;
extracting a movement data value from the movement data;
looking up an audio-visual data file comprising data representing audible corrective instructions if the movement data value is outside a pre-determined movement range.

13. The method according to claim 1, wherein executing the algorithm for determining a heartbeat further comprises, based on at least two heartbeats, determining at least one of a heartrate and a heart rhythm.

14. A handheld device for determining a heartbeat or heart rhythm of a user, and for providing spoken instructions for the user of the device, the device comprising:
an optical sensor arranged to be brought in proximity of a member of a body of the user for obtaining, over time, a data signal;
an electronic data processor arranged to:
determine, based on the signal, a quality factor for the signal;
execute an algorithm for determining a heartbeat using data comprised by the data signal as input;
look up, from an electronic memory, at least one audio-visual data file comprising data representing audio-visual feedback instructions, based on at least one of the quality factor and the outcome of the execution of the algorithm; and
a speaker for reproducing the audio-visual feedback instructions.

15. A non-transitory computer readable medium comprising a program of instructions that, when executed by a processor, perform the method according to claim 1.
